(19) United States
(12) Patent Application Publication
(10) Pub. No.: US 2008/0208315 A1
(43) Pub. Date: Aug. 28, 2008

(54) Coronary stent having a surface of multi-layer immobilized structures

(75) Inventors: Ming-Chien Yang, Taipei City (TW); Juey-Jen Hwang, Taipei City (TW); Li-Ying Huang, Taipei City (TW); Ting-Yu Liu, SinfongTownship (TW)

Correspondence Address: BIRCH STEWART KOLASCH & BIRCH, PO BOX 747, FALLS CHURCH, VA, 22040-0747, US

(73) Assignee: National Taiwan University of Science & Technology

(21) Appl. No.: 11/710,999

(22) Filed: Feb. 27, 2007

(51) Int. Cl. A61F 2/06

(52) U.S. Cl. 623/1.15

(57) ABSTRACT

A stent for coronary vessels, having a surface of multilayer immobilized structures, includes a stent body and a number of polyelectrolyte complex (PEC) layers stacking and being immobilized on the surface of the stent body, in which the PEC layer is formed of a polymer layer and an anticoagulant layer. The coronary stent is capable of effectively improving the hemocompatibility longevity over conventional stent using surface encapsulation of an anticoagulant layer for hemocompatibility improvement. Furthermore, the coronary stent can be use as a drug-eluting coronary stent, thus allowing for the time-releasing of drugs, and further preventing the thickening of vascular smooth muscle cells for causing vascular thrombosis.

### BACKGROUND OF THE INVENTION

1. Field of the Invention

The present invention relates in general to a stent for blood vessels, and in particular to a stent for coronary vessels having a surface of multilayer immobilized structures.

2. The Prior Arts

Coronary stent currently has been widely adopted in connection with percutaneous transluminal coronary angioplasty (PTCA) for use in the treatment of coronary arterial diseases. The coronary stent is mainly used in emergency percutaneous coronary intervention; therefore, the most important function of the implanted coronary stent is to prevent restenosis. On the other hand, the problem of stent thrombosis when using conventional coronary stent is very severe, even to the extent of having 2-5% of patients suffering from subacute stent thrombosis.

Conventional stent typically is made from stainless-steel (SS), and is used for opening blocked blood vessels. However, the conventional stainless-steel stent does not possess adequate hemocompatibility; therefore, many researchers have attempted to improve the hemocompability of stainless-steel stent.

Used as a strategy for preventing the nonspecific absorption of blood constituents, a polysaccharide material is used to coat the stent. Furthermore, this is because the conventional polysaccharide coating can prevent the nonspecific absorption of the protein. For example, Thierry et al. had previously disclosed a type of self-assembled polyelectrolyte multilayer of hyaluronic acid and chitosan, which are coated on the surface of the NiTi stent. The aforementioned structure contains sodium nitroprusside, and can reduce platelet adhesion by 40% (B. Thierry, F. M. Winnik, Y. Merhi, J. Silver, M. Tabrizian, Biomacromolecules 4, 1564 (2003)). Yoshioka et al. have disclosed a laminaria japonica layer coated on the stainless-steel substrate for reducing the platelet adhesion by over 70% (T. Yoshioka, K. Tsuru, S. Hayakawa and A. Osaka, Biomaterials 24, 2889 (2003)).

However the ability to improve the effectiveness for hemocompatibility of using only polysaccharide for coating the stainless-steel coronary stent is still rather limited, as this type of coronary stent cannot completely resolve the thrombus issue. As a result, some have proposed to add heparin (HEP), dextran, aspirin, or some cytostatics, such as, for example, concentrated treatments using sirolimus for the improvement of the hemocompatibility of conventional stent, which uses only polysaccharide coating to the stent. For example, the bioactivity of the conventional grafted heparin can make the polymer containing heparin permanently on the surface to thus improve the original hemocompatibility. However, the conventional heparin cannot be securely disposed on the surface of the polymer. Therefore, for the preparation of a more rigid anticoagulant surface, there are already many researchers attempting to incorporate the sulfonate groups to modify the compositions of the polymer material, which is the so-called heparin-like material (heparinoid). However, this type of slight modification produces yet still unsatisfactory results; the heparin that is secured on the stent still would be released quickly, thus making the stent to lose its anticoagulant effectiveness after implanting into living tissues.

As a result, an important objective is to develop a new coronary stent system capable of functioning as a drug-eluting coronary stent system.

### SUMMARY OF THE INVENTION

A primary objective of the present invention is to provide a stent for coronary vessels having a surface of multilayer immobilized structures, which is able to overcome the issues and problems of conventional technology, and to improve the hemocompatibility of the conventional coronary stent, and also further providing functionality as a drug-eluting coronary stent.

According to the present invention, a coronary stent having a multilayer immobilized structure is proposed, which includes the following: a stent body, and a plurality of polyelectrolyte complex (PEC) layers stacking and being immobilized on the surface of the stent body, wherein the polyelectrolyte complex (PEC) layer is formed of a polymer layer and an anticoagulant layer.

According to the present invention, a coronary stent capable of effectively improving the hemocompatibility longevity over the conventional stent using surface encapsulation of an anticoagulant layer for hemocompatibility improvement is provided. Furthermore, the coronary stent of the present invention can be configured for use as drug-eluting coronary stent, thereby allowing for the time-release of drugs.

Although the present invention has been described with reference to the preferred embodiments below, it is apparent to those skilled in the art that a variety of modifications and changes may be made without departing from the scope of the present invention which is intended to be defined by the appended claims.

### BRIEF DESCRIPTION OF THE DRAWINGS

The present invention will be apparent to those skilled in the art by reading the following detailed description of a preferred embodiment thereof, with reference to the attached drawings, in which:

FIG. 1 is a cross-sectional view of a coronary stent, according to a preferred embodiment of the present invention;

FIG. 2 is an illustrative schematic of an enlarged section as denoted in the shaded region in FIG. 1;

FIG. 3 is a schematic illustrating test results for a plurality of samples in contact angles using goniometer;

FIGS. 4(A)-(C) are a plurality of XPS charts for pure SS, SS-ATMS, SS-ATMS-HA, and SS-ATMS-HA-HEP substrate samples, wherein FIG. 4(A) is for Si₂ₚ; FIG. 4(B) is for N₁ₛ; and FIG. 4(C) is for S₂ₚ;

FIG. 5 is a chart illustrating the analytical results of the stabilized HEP on pure-SS and the APTT, PT, FT and TT for the SS-ATMS-HA-HEP;

FIG. 6(A) is an analytical chart illustrating the cumulated released rate of sirolimus from the samples; and

FIG. 6(B) is an analytical chart illustrating the corresponding results using equations (2) and (3) and data from FIG. 6(A).

### DETAILED DESCRIPTION OF THE PREFERRED EMBODIMENT

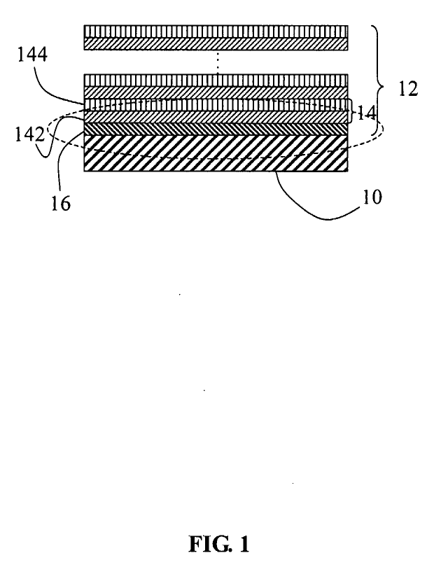

With reference to the drawings and in particular to FIG. 1, a coronary stent according to a preferred embodiment of the present invention is presented. In accordance with the present invention, the coronary stent includes a stent body 10 and a multilayer immobilized structure 12, which is formed on the stent body 10. The multilayer immobilized structure 12 is formed of a plurality of polyelectrolyte complex layers 14, and each polyelectrolyte complex layer 14 is separately formed of a polymer layer 142 and an anticoagulant layer 144. In other words, the aforementioned multi-layer immobilized structure 12 is formed by the repetitive stacking of the polymer layer 142 and the anticoagulant layer 144.

The aforementioned multilayer immobilized structure can be formed based upon the desired thickness required using the repetitive stacking of the polyelectrolyte complex layers. Therefore, in theory, there is no maximum limit to the number of layers that can be formed. However, based upon manufacturing cost and efficiency considerations, the polyelectrolyte complex layer 14 is preferred to be kept at between 2 to 20 layers, in which a more preferred embodiment is at 2 to 10 layers. According to the present invention, the thickness of each polyelectrolyte complex layer is on the order of nanometer scale.

The stent body, as described according to the present invention, can be fabricated using any conventional method and any conventional material for fabricating a coronary stent. For example, the material for the coronary stent body can be, for example, stainless-steel, but is not limited as such.

The polymer compound which can be utilized in the polymer layer according to the present invention, as long as conventionally known of comprising of biocompatibility, biodegradability, and with polymer material of having negative charge groups and bases, can all be applicable for use in the present invention, thus no special limitations are being provided according to the present invention. Some examples include, but are not limited to, hyaluronic acid, chondroitin sulfate, alginic acid, and bovine serum albumin, etc.

The aforementioned anticoagulants do not include any particular limitations. Basically, any conventional anticoagulant can be utilized in the present invention, such as, but is not limited to, heparin.

The anticoagulant layer according to the present invention is preferred to be formed by chemical bonding on the polymer layer. And, the conventional polymer material is not easily secured on the stainless steel; therefore when the stent body 10 is made of stainless steel, the polymer layer 142 according to the present invention preferably is to use a stent adhesion layer 16 acting as a bridge for coupling the stainless steel surface. Examples of the stent adhesives which can be applicable for the present invention are, such as, a silane or a thiol having an amino group. An example of the aforementioned silane is but not limited to aminotrimethoxysilane (ATMS). An example of the aforementioned thiol is but not limited to dimercaptosuccinic acid (DMSA).

The coronary stent according to the present invention can further encapsulate a therapeutic drug, such as, for example, anti-inflammatory drug, anticoagulant, cell growth inhibitor, but is not limited to the above. An example of a cell growth inhibitor is Rapamune® (sirolimus), which is a type of conventional immunosuppressant, which is using Streptomyces hygroscopicus to form macrocyclic lactone. Sirolimus uses a different mechanism from other immunosuppresant for supressing the T-cell activity and growth that are triggered by reacting antigen and cellular stimulation. Therefore, sirolimus can suppress antibody formation.

The conventional hyaluronic acid is a linear polysaccharide formed from a type of repetitive disaccharide unit for the N-acetylglucosamine along with d-glucuronic acid. Because the hyaluronic acid contains an extracellular matrix (ECM); therefore, it has very high lubricity, water-sorption, and water retention capabilities. In addition, it especially can affect several types of cellular functions, such as attachment, migration, and proliferation. As a result, the stent, according to the present invention, includes superior biocompatibility.

As the stent, according to an embodiment of the present invention, is implanted into a biological body, the anticoagulant that is exposed at the outermost layer (for example, heparin) shall effectively suppress the coagulation of the blood platelet. Although the anticoagulant at the outermost layer should slowly be spent, but as the implant duration is increased, the subsequent layer of polymer layer, which is biodegradable, shall slowly be degraded. And the anticoagulant layer underneath the polymer layer is permitted to be exposed. Therefore, the anticoagulant on the stent is then continuously released, thereby producing the effect of anticoagulation.

Although the present invention has been described with reference to the preferred embodiment thereof, it is apparent to those skilled in the art that a variety of modifications and changes may be made without departing from the scope of the present invention which is intended to be defined by the appended claims.

### First Embodiment

### Apparatus for Coronary Stent

First, surface modification is performed on a stainless-steel plate (SUS316L). The stainless steel plate, prior to being heated at 500° C., is firstly ultrasonic-vibrated three times inside an acetone solution, and later is soaked in nitric acid for 20 minutes, thereby removing the impurities from the stainless-steel surface. The stainless steel plate after the aforementioned cleaning process is hereby referred to as “pure SS”.

The stainless-steel plate using aminotrimethoxysilane (ATMS) is taken to perform silylation, wherein the stainless-steel plate is soaked inside 1 wt % ATMS toluene solution, and also agitation under ultrasound for one hour is performed. After cleaning using toluene and ethanol, finally again sonic vibration is performed for 5 minutes. Then it is air dried. The sample produced is thereby referred to as “SS-ATMS”.

The “SS-ATMS” stainless steel plate is then soaked at 25° C. in 20 ml distilled water containing 0.5 g of hyaluronic acid (HA) and 0.3 g of 1-ethyl-3-(3-dimethylaminopropyl)carbodiimide (EDC) for two hours. Severe agitation is performed to allow the (—NH₂) group of the ATMS to be coupled to the (—COO⁻) group of the hyaluronic acid for forming the amide linkages. Thereafter, the sample is to be further cleaned using distilled water, and is ultrasonic vibrated for 5 minutes inside the distilled water. Hereafter, the obtained sample is thereby referred to as “SS-ATMS-HA”.

Later, the “SS-ATMS-HA” thin plate is be soaked at 4° C. for 24 hours in 0.01 M EDC solution. The sample is further cleaned three times total, including once using phosphate buffer saline (PBS) and twice using distilled water for removing the residual EDC. Later, the substrate, after being EDC-activated, is put into a citric acid solution of the 2000 IU/ml HEP at 4° C. for 24 hours for allowing the (—OH) functional group of the HEP to be coupled to the (—COO⁻) group of the HA, thus forming an ester bond. The sample is to be cleaned three times total, where once using PBS and twice using distilled water. The obtained sample is air dried for 24 hours at 25° C. The aforementioned sample is hereby referred to as “SS-ATMS-HA-HEP” (or as “a layer of HA-HEP”).

Later, the aforementioned procedures are repeated 1, 3, and 5 times. The resulting sample is hereby referred to as one-layer HA-HEP, three-layer HA-HEP, and five-layer HA-HEP.

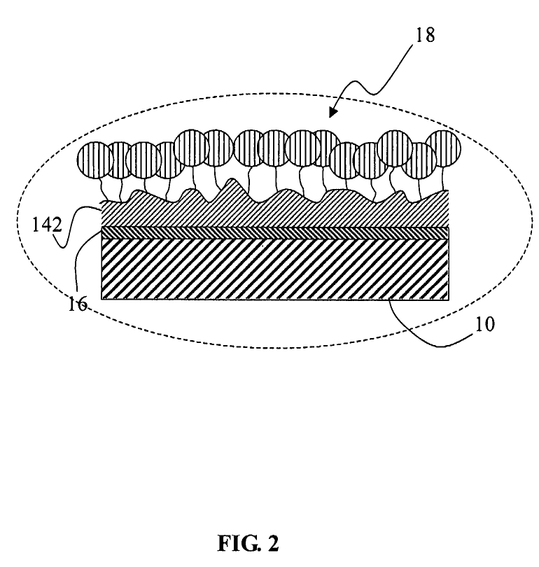

FIG. 2 is an illustrative schematic of a brush 18 having hyaluronic acid that is secured on the stainless steel surface fabricated by means of the aforementioned method. The brush 18 portion is formed from heparin via chemical bonding to couple to the hyaluronic acid.

### Second Embodiment

The stent sample which has underwent surface modification as fabricated in the first embodiment has its surface hydrophilicity measured using a goniometer and assessed using the water contact angle (θ). The stainless steel sample, after surface modification, is using Mg as the anode, and is analyzed under XPS at 1253.6 eV and 150 W of power. Scanning measurements are performed using a variety of eVs to O₁ₛ, Si₂ₚ, N₁ₛ, and S₂ₚ. The surface roughness is inspected using atomic force microscopy (AFM).

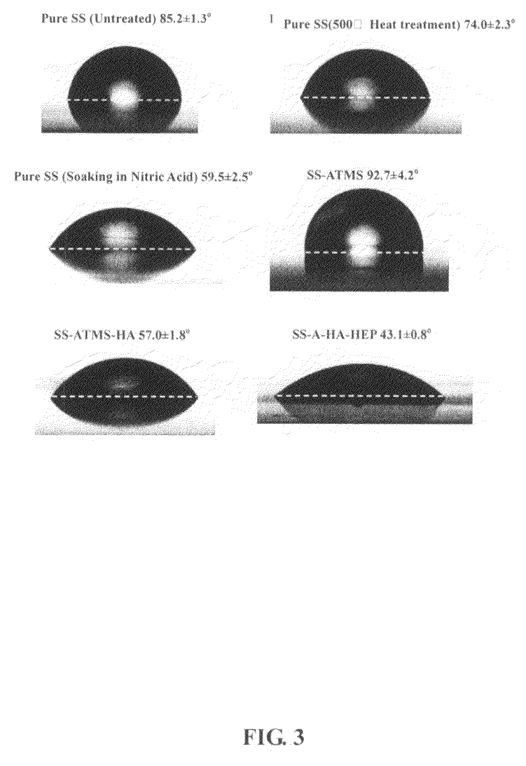

FIG. 3 illustrates the contact angles between the surface-modified stainless steel samples and distilled water. After performing heat treatment (74.0°) and nitrate immersion (59.5°) at 500° C., the contact angles are lower than the untreated stainless steel sample (85.2°), and later the silylation clearly changes the surface wettability. Wherein is visible after silylation is to be embedded in the HA and HEP steps (namely, SS-ATMS-HA and SS-ATMS-HA-HEP) when compared with SS-ATMS, it is clearly evident that the contact angles are reduced (53.0° and 43.7°). This result supports the fact that some hydrophilic groups (for example, HA and HEP) are already subsequently embedded on the SS-ATMS substrate. This is evidenced by the test results using the XPS.

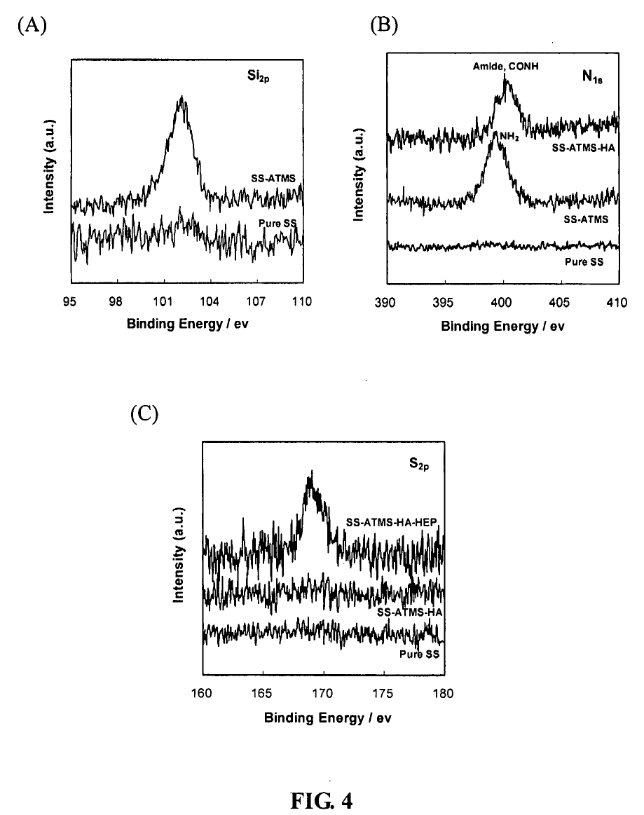

FIGS. 4(A)-4(C) show the comparison of XPS charts for pure SS, SS-ATMS, SS-ATMS-HA, and SS-ATMS-HA-HEP substrate materials. The XPS analysis results indicated that the surface of the substrate is mainly composed of Si, N, and S atoms. FIG. 4(A) shows the XPS data for the Si₂ₚ in pure SS and SS-ATMS substrate. The peak in the SS-ATMS can be found to be 102.0 eV, thus implying that the silane in the ATMS is already anchored on the SS piece. In addition, the (NH₂—) peak value (399.4 eV) of the SS-ATMS can be observed in the N₁ₛ scan chart, and this indicates that the ATMS is already anchored on the stainless-steel substrate, as illustrated in FIG. 4(B). However, when the HA-COO⁻ and the ATMS-NH₂ are bonded, the amino peak is shifted from 399.4 eV to 400.2 eV, which indicates the formation of the (—CONH). Furthermore, in comparison to the pure SS, the SS-ATMS-HA, and the SS-ATMS-HA-HEP substrate materials, the S₂ₚ peak value for the HEP ((binding energy) 168.7 eV) can be observed in the spectra for the SS-ATMS-HA-HEP substrate material, as illustrates in FIG. 4(C). The aforementioned result confirms that the ATMS, HA, and the HEP have effectively bonded on the stainless steel plate.

Table 1 provides the explanation of the surface roughness and outer appearance condition for the samples after surface modification by means of AFM inspection. Initially, the exposed stainless steel exhibits a grain-like structure and a flatter surface (Ra: 8.16 nm). However, due to the bonding of ATMS, the surface becomes roughened (Ra: 14.35 nm). When the HA/HEP PEC is secured onto the surface of the SS-ATMS, the obtained image displays an even more roughened structure (also known as nanobrush structure). Among these substrate materials, the roughness shall be increased proportional to the number of encapsulated layers. When comparing a one-layer HA-HEP with a three layer HA-HEP, it is evident that the surface roughness of the three-layer sample (Ra: 36.66 nm) is higher than that of the one-layer sample (Ra: 20.19 nm), this indicates that the HA/HEP PEC can be effectively accumulated. However, the roughness of the five-layer sample is only slightly increased (Ra: 37.98 nm). And we can find that the surface structure of the five-layer HA-HEP is more compact or dense than the one-layer HA-HEP and the three-layer HA-HEP. These results indicate that, the five-layer encapsulation may lead to reduced porosity and a more compact structure; and these different structures shall lead to different drug time-release rates.

Table 1 indicates that the thickness of the HA/HEP PEC layer is between 280 to 630 nm, and this indicates that the obtained sample is of nanoscale structure. However, the thickness is not directly proportional to the number of layers. This may be caused by the fill up of the “valleys” of the previous layer. In other words, the HA/HEP PEC for the subsequent layer, apart from bonding on the tip region of the brush, is also bonded on the inclined portion of the brush.

### Third Embodiment

30 ml of human blood is retrieved from a healthy donor and is mixed with a liquid solution containing 0.136 M D-glucose, 75 mM sodium citrate, and 0.4 mM citric acid. Later, the human blood is centrifuged under 300 g at 4° C. for 20 minutes for separating the blood corpuscles from the platelet-rich plasma (PRP). Later, a portion of the PRP is removed and centrifuge is performed under 2000 g at 4° C. for 20 minutes to obtain the platelet-poor plasma (PPP) to provide for the human plasma-protein adsorption test. The substrate material is placed in 0.5 ml of PPP at under 37° C. for 1 hour. The activated partial thrombin time (APTT) for the PPP undergoing reaction, the prothrombin time (PT), the fibrinogen time (FT), and the thrombin time (TT) are measured using an automated blood coagulation analyzer during testing. In addition, testing is performed to test tubes containing no test samples as the control group.

The blood coagulation cascade includes intrinsic pathway, extrinsic pathway, and common pathway. Among these blood clotting time periods, APTT is mainly related to intrinsic pathway and common pathway, PT is mainly related to extrinsic pathway and common pathway, and FT and TT are used for the detection of the duration for transforming of fibrinogen into fibrin.

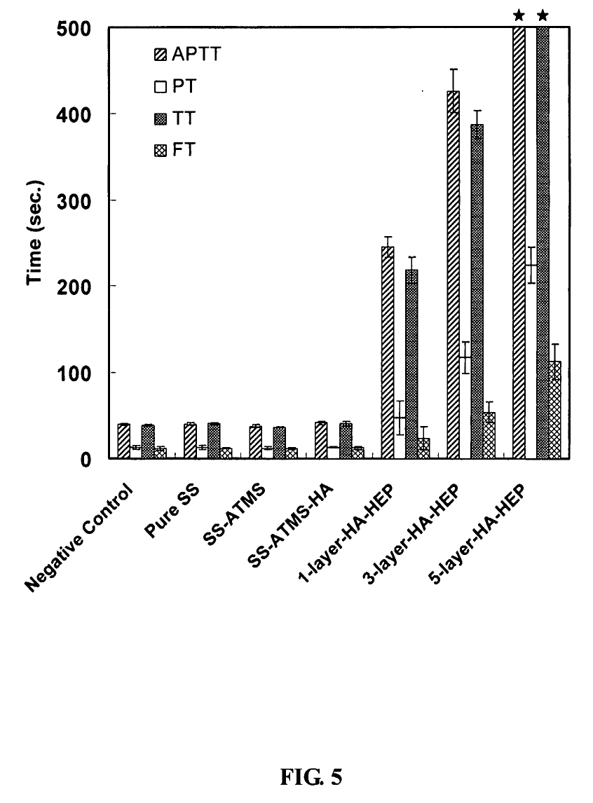

FIG. 5 and Table 2 indicate the effects of stabilized HEP on pure-SS and the APTT, PT, FT and TT for the SS-ATMS-HA-HEP. A stable heparin can activate ATIII, and thus in turn prevents thrombus formation. The results indicate that the APTT, PT, FT and TT for SS-ATMS-HA-HEP is individually 6.1, 3.7, 1.2, and 5.4 times of that for the pure SS. The blood clotting time periods of the pure SS is closer to human plasma (negative control group), indicating that the pure-SS does not possess anticoagulant activity. Furthermore, the SS-ATMS has slightly lower blood clotting time periods than pure SS. The reason is that the (—NH₂) functional group of the ATMS can stimulate the activation of the platelet, and therefore reducing the blood clotting time periods. On the other hand, the SS-ATMS-HA sample has longer blood clotting time periods, and therefore has reduced blood coagulation. The quantity of encapsulation layers shall affect the anticoagulation activity. This result indicates that the blood clotting time periods shall be increased proportionally to the number of encapsulation layers. The maximum anticoagulation activity is observed for the five-layer HA-HEP sample, in which the APTT and the TT are both in excess of 500 s (which is at the upper limit for the coagulation analyzer), thus showing the superior anticoagulant activity of the sample.

### Fourth Embodiment

### Drug Time-Release Testing

In in vitro drug time-release testing, the sample made from the first embodiment is to be placed in the saturated liquid solution of ethanol of the sirolimus, and is agitated for 24 hours under 4° C. at 100 rpm speed, thereby allowing the sirolimus to be embedded into the multiple-layers of the stent surface. Later, the stent with sirolimus embedded is taken to conduct drug time-release testing in 5 ml of buffer solution at 37° C. At a predetermined time interval, the buffer solution is removed and later the appropriate dilution is prepared, where the drug time-release concentration is tested via the UV spectra at 231.6 nm. The drug time-release rate in percentage is determined from Equation (1) below:

$$Accumulated drug time \text{-} release rate ( \% ) = \frac{{R}_{t}}{L} \times 100 \% \qquad ( 1 )$$

where L and Rₜ are, respectively, the initial drug loading and the accumulated drug time-release amount at time t.

For studying the dispersion mechanism of colloids, the drug time-release information is further taken using Equation (2) below:

$$\frac{{M}_{t}}{M} = {kt}^{″} \qquad ( 2 )$$

where Mₜ is the mass of sirolimus released at time t, M is the mass of sirolimus released at infinite time, and Mₜ/M is the mass fraction of the time-released sirolimus; k is a characteristic constant, and n is the characteristic exponent relating to the penetrant transport. By taking the logarithm on both sides of Equation (2), Equation (3) is provided for calculating the dispersion parameters (n and k) when Mₜ/M<0.6:

$$ln ( \frac{{M}_{t}}{M} ) = n ln t + ln k \qquad ( 3 )$$

As during the time t and at the termination of the experiment (approaching infinite time), the accumulated concentration of the time-released sirolimus is used for calculating Mₜ/M.

Table 1 lists the loading and loading efficiency of the sirolimus in SS-ATMS-HA/HEP nanobrush. The sirolimus (encapsulated) rate is respectively accordingly as five-layer HA-HEP>three-layer HA-HEP>one-layer HA-HEP nanobrush. The five-layer HA-HEP loading efficiency is therefore higher than the one-layer HA-HEP and the three-layer HA-HEP. The possible reason for the five-layer HA-HEP to have a higher loading efficiency is possibly due to it having a thicker structure.

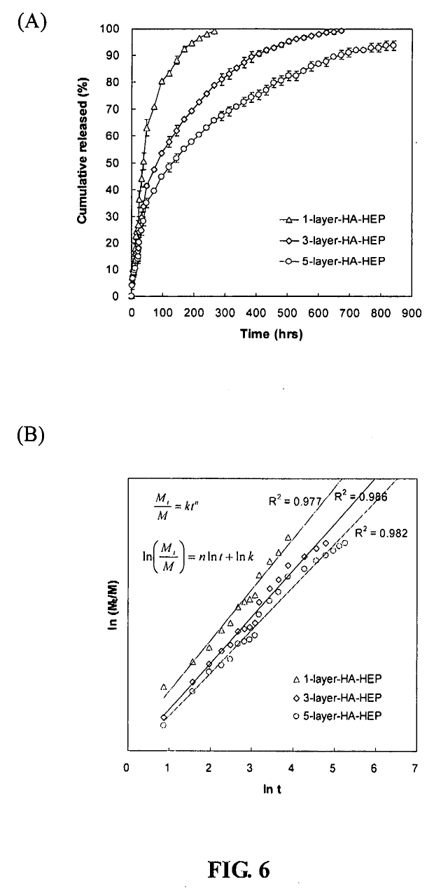

FIG. 6(A) illustrates the accumulated time-release chart of sirolimus from the sample. In particular, the sirolimus can be time-released from the five-layer HA-HEP sample in excess of 30 days. The time requirement for the complete time-release of the sirolimus shall be increased as the number of the encapsulation layers is increased (three-layer HA-HEP can time-release at least approximately 26 days, and a one-layer HA-HEP can time-release at least approximately 10 days). This is possibly because the five-layer HA-HEP sample possesses more compact porosity, which is already evidenced in the AFM photo, and thus leading to the reduction of the drug time-release rate and the extension of the time-release duration.

Some researchers have segregated three types of dispersion release mechanisms from the swellable controlled release system. The first type is Fickian diffusion (n=0.5), where the dispersion rate is far less than the relaxation rate. Under this type of mechanism, the time-release system is using dispersion for control. The second type of mechanism is the Case II transport (n=1.0), where the dispersion process far exceeds the relaxation process. This control step is performed at the advancing front speed, in which the said front is forming a boundary between the inflated colloid and the glassy core. The third type is an anomalous (non-Fickian) transport (n=0.5-1.0), in which the situation where the dispersion and relaxation speed are equal is described.

The parameters n and k are calculated from Equations (2) and (3), and is listed in Table 1 and FIG. 6(B). The n value for the one-layer HA-HEP, the three-layer HA-HEP and the five-layer HA-HEP is 0.748, 0.682, and 0.630, respectively. All of the transports for the 1 to 5 encapsulation layers are non-Fickian diffusion, with having dispersion and relaxation control system. Furthermore, the k value in these HA/HEP PEC encapsulated substrate materials is respectively accordingly: one-layer HA-HEP (3.10)>three-layer HA-HEP (2.52)>five-layer HA-HEP (2.43). These results indicate that the time-release rate for the sirolimus decreases as the number of encapsulation layers is increased.

As can be determined from the aforementioned results, the bonded heparin can reduce the adhesion of the platelet when coming into contact with the blood; therefore, the activation of the anticoagulation cascade is prevented. The bonded heparin can also activate AT III, which therefore in turn suppresses the prothrombin from becoming thrombin. The two mechanisms both can suppress coagulation cascade.

TABLE 1
Surface roughness and drug time-release parameters (k and n)
for the stainless-steel stent after surface modification.

| | | | Drug | | | |
|---|---|---|---|---|---|---|
| | Surface | | Loading | | | |
| | Roughness | Drug Loading^{b} | Efficiency | | | Thickness^{d} |
| | (Ra, nm)^{a} | (μg/cm²) | (%) | k^{c} | n^{c} | (nm) |
| Pure SS | 8.16 ± 0.5 | — | — | — | — | — |
| SS-ATMS | 14.35 ± 0.8 | — | — | — | — | — |
| 1-laycr-HA-HEP | 20.19 ± 1.5 | 1.02 ± 0.04 | 19.92 | 3.10 | 0.748 | 280 ± 3.6 |
| 3-layer-HA-HEP | 36.66 ± 2.1 | 1.96 ± 0.09 | 38.28 | 2.52 | 0.682 | 480 ± 8.1 |
| 5-layer-HA-HEP | 37.98 ± 1.2 | 3.12 ± 0.15 | 60.94 | 2.43 | 0.630 | 630 ± 5.1 |

NOTE:
^{a}AFM is used to inspect the surface roughness (Ra) (n = 5)
^{b}drug loading (mg/cm² - S-ATMS-HA/HEP nanobrush) (n = 5)
^{c}k and n value calculated based upon Equation (3)
^{d}Membrane thickness determined using spectroscopic ellipsometer (n = 5)

TABLE 2
Blood clotting time periods for stainless-steel
stents after surface modification, including APTT, PT, FT, and TT.

| | APTT | PT | TT | FT |
|---|---|---|---|---|
| negative | 39.2 ± 1.2 | 12.6 ± 2.1 | 38.7 ± 1.4 | 12 ± 2.1 |
| control group | | | | |
| Pure SS | 40.1 ± 1.8 | 12.7 ± 2.6 | 40.9 ± 1.6 | 11.9 ± 0.7 |
| SS-ATMS | 37.65 ± 1.5 | 12.05 ± 1.5 | 36.5 ± 0.9 | 11.6 ± 1.5 |
| SS-ATMS-HA | 41.8 ± 1.8 | 13.3 ± 0.9 | 41.1 ± 2.4 | 12.5 ± 1.8 |
| 1-layer-HA- | 245.8 ± 12 | 47.5 ± 20 | 218.9 ± 15 | 23.8 ± 13 |
| HEP | | | | |
| 3-layer-HA- | 425.7 ± 25 | 117.6 ± 1 | 387 ± 16 | 54 ± 12 |
| HEP | | | | |
| 5-layer-HA- | No clotting | 225 ± 21 | No clotting | 112.5 ± 20 |
| HEP | | | | |

What is claimed is:

1. A coronary stent having a surface of multi-layer immobilized structures, comprising:
a stent body; and
a plurality of polyelectrolyte complex layers stacking and being immobilized at the surface of the stent body, wherein the polyelectrolyte complex layer is formed of a polymer layer and an anticoagulant layer.

2. The coronary stent as claimed in claim 1, wherein the number of layers of the polyelectrolyte complex layer is between 2 to 20 layers.

3. The coronary stent as claimed in claim 1, wherein the stent body is made of stainless steel.

4. The coronary stent as claimed in claim 1, wherein the polymer layer is selected from the group consisting of hyaluronic acid, chondroitin sulfate, alginic acid, and bovine serum albumin.

5. The coronary stent as claimed in claim 1, wherein the anticoagulant in the anticoagulant layer is heparin.

6. The coronary stent as claimed in claim 1, further comprising a stent adhesion layer between the polyelectrolyte complex layer and the stent body.

7. The coronary stent as claimed in claim 6, wherein the stent adhesive forming the stent adhesion layer is comprised of an amino group and is silane or thiol.

8. The coronary stent as claimed in claim 7, wherein the silane having an amino group is aminotrimethoxysilane.

9. The coronary stent as claimed in claim 7, wherein the thiol is dimercaptosuccinic acid.

10. The coronary stent as claimed in claim 1, wherein a therapeutic drug is further encapsulated within the polyelectrolyte complex layer.

11. The coronary stent as claimed in claim 10, wherein the therapeutic drug is an anti-inflammatory drug, an anticoagulant, or a cell growth inhibitor.

12. The coronary stent as claimed in claim 11, wherein the cell growth inhibitor is sirolimus.
